(19) United States
(12) Patent Application Publication
(10) Pub. No.: US 2023/0012942 A1
(43) Pub. Date: Jan. 19, 2023

(54) HEALTH CONDITION ESTIMATION APPARATUS AND HEALTH CONDITION ESTIMATION METHOD

(71) Applicant: KYOCERA CORPORATION, Kyoto-shi, Kyoto (JP)

(72) Inventor: Shinichi ABE, Uji-shi, Kyoto (JP)

(21) Appl. No.: 17/783,158

(22) PCT Filed: Dec. 7, 2020
(86) PCT No.: PCT/JP2020/045543

§ 371 Date: Jun. 7, 2022

(30) Foreign Application Priority Data
Dec. 10, 2019 (JP) 2019-223119

(51) Int. Cl. G16H 50/30; G01N 33/497

(52) U.S. Cl. CPC G16H 50/30; G01N 33/497

(57) ABSTRACT

A health condition estimation apparatus 10 includes an obtaining unit 40 and a controller 42. The obtaining unit 40 obtains gas information. The gas information is based on a signal output by a sensor unit in a period in which a first gas is supplied to the sensor unit and a signal output by the sensor unit in a period in which a second gas is supplied to the sensor unit. The sensor unit outputs a signal with a signal value in accordance with the concentration of a specific gas. The first gas and the second gas are different in at least either of obtaining position and obtaining time. A controller 39 generates health information based on the gas information.

### CROSS-REFERENCE TO RELATED APPLICATIONS

The present application claims priority from Japanese Patent Application No. 2019-223119 filed on Dec. 10, 2019, the entire contents of which are hereby incorporated by reference.

### TECHNICAL FIELD

The present invention relates to a health condition estimation apparatus and a health condition estimation method.

### BACKGROUND ART

It has been reported that the intestinal environment, especially resident intestinal bacteria, is heavily involved in human health maintenance, disease prevention, and the like. For example, it has been proposed to analyze a subject's feces to determine the deviation of resident intestinal bacteria from a preferable type of resident intestinal bacteria obtained on the basis of a correlation set in advance on the basis of the subject's diet, and to seek a diet for realizing the preferable type of resident intestinal bacteria (see PTL 1 below).

### CITATION LIST

### Patent Literature

PTL 1: Japanese Unexamined Patent Application Publication No. 2012-165716

### SUMMARY OF INVENTION

A health condition estimation apparatus according to a first aspect includes:

an obtaining unit that obtains, from a sensor unit that outputs a signal with a signal value in accordance with a concentration of a specific gas, to which a first gas and a second gas are supplied, the first gas and the second gas being different in at least either of obtaining position and obtaining time, gas information based on a signal output by the sensor unit in a period in which the first gas is supplied to the sensor unit and a signal output by the sensor unit in a period in which the second gas is supplied to the sensor unit; and

a controller that generates health information using the gas information.

A health condition estimation method according to a second aspect of the present disclosure includes:

separately supplying a first gas and a second gas to a sensor unit that outputs a signal with a signal value in accordance with a concentration of a specific gas, the first gas and the second gas being different in at least either of obtaining position and obtaining time; and

generating health information based on gas information including a signal output by the sensor unit in a period in which the first gas is supplied to the sensor unit and a signal output by the sensor unit in a period in which the second gas is supplied to the sensor unit.

### BRIEF DESCRIPTION OF DRAWINGS

FIG. 1 is a configuration diagram illustrating a schematic configuration of a health condition estimation system including a server serving as a health condition estimation apparatus according to a present embodiment.

FIG. 2 is an external perspective view illustrating an installing mode of a fecal odor measuring apparatus in FIG. 1.

FIG. 3 is a functional block diagram schematically illustrating the internal configuration of the fecal odor measuring apparatus in FIG. 1.

FIG. 4 is a configuration diagram schematically illustrating the configuration of the fecal odor measuring apparatus in FIG. 1.

FIG. 5 is a functional block diagram schematically illustrating the internal configuration of a terminal apparatus in FIG. 1.

FIG. 6 is a functional block diagram schematically illustrating the internal configuration of the health condition estimation apparatus in FIG. 1.

FIG. 7 is an external view illustrating an example in which health information generated by the terminal apparatus in FIG. 1 is displayed on a display unit.

FIG. 8 is a first flowchart for describing a gas information generating process executed by a controller of the fecal odor measuring apparatus in FIG. 1.

FIG. 9 is a second flowchart for describing the gas information generating process executed by the controller of the fecal odor measuring apparatus in FIG. 1.

FIG. 10 is a flowchart for describing a physical information etc. giving process executed by a controller of the terminal apparatus in FIG. 1.

FIG. 11 is a flowchart for describing an intestinal status giving process executed by the controller of the terminal apparatus in FIG. 1.

FIG. 12 is a flowchart for describing a health information generating process executed by a controller of the health condition estimation apparatus in FIG. 1.

FIG. 13 is a flowchart for describing an estimation formula updating process executed by the controller of the health condition estimation apparatus in FIG. 1.

### DESCRIPTION OF EMBODIMENTS

Hereinafter, an embodiment of a health condition estimation apparatus to which the present invention is applied will be described with reference to the drawings.

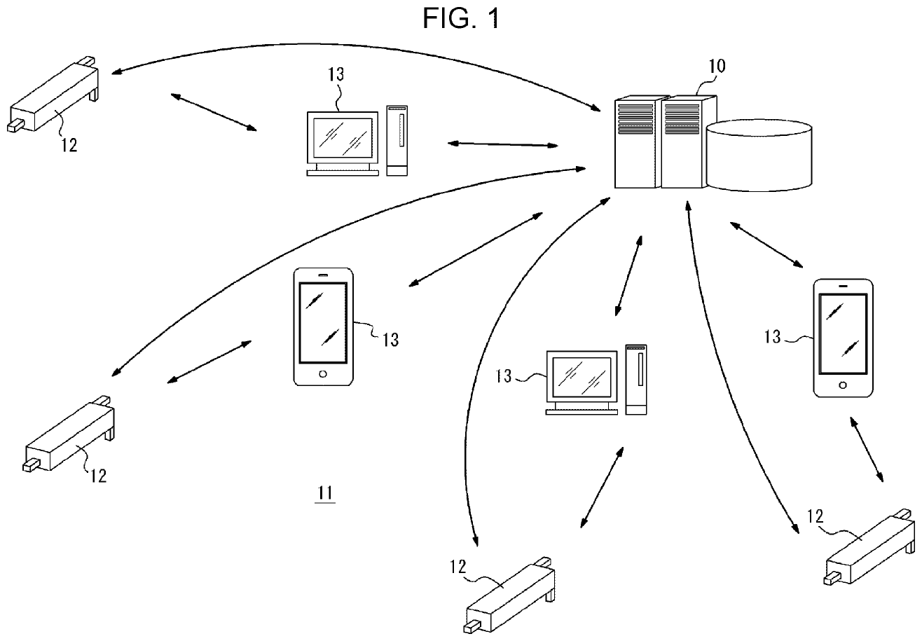

A health condition estimation system 11 including a health condition estimation apparatus 10 according to an embodiment of the present invention includes, as illustrated in FIG. 1, a plurality of sets of fecal odor measuring apparatuses 12 and terminal apparatuses 13, and the health condition estimation apparatus 10.

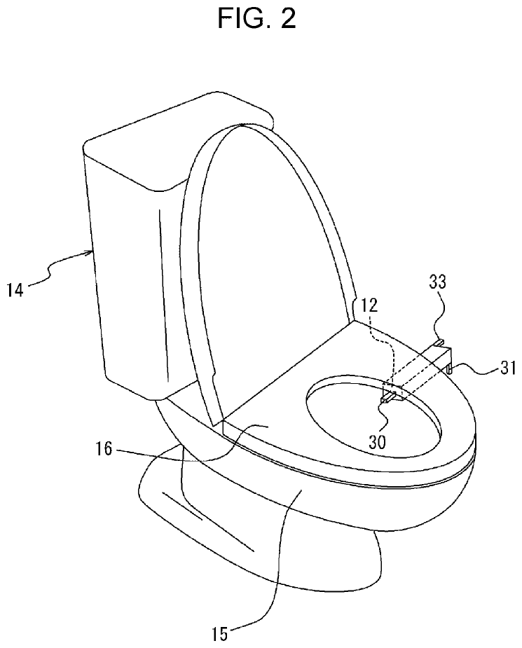

As illustrated in FIG. 2, each fecal odor measuring apparatus 12 is installed in, for example, a flush toilet 14. The fecal odor measuring apparatus 12 may be installed at any place in the toilet 14. For example, the fecal odor measuring apparatus 12 may be arranged from between a toilet bowl 15 and a toilet seat 16 to the outside of the toilet 14. The fecal odor measuring apparatus 12 may be partially embedded in the toilet seat 16. In addition, the fecal odor measuring apparatus 12 includes a first inlet 30, a second inlet 31, and an outlet 33.

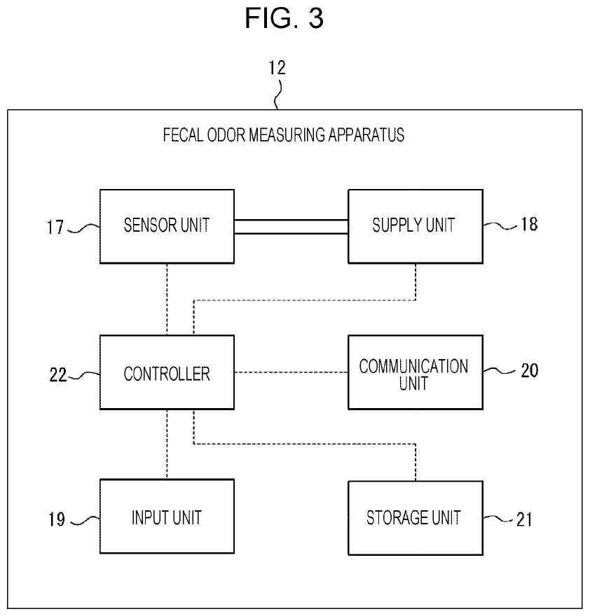

As illustrated in FIG. 3, the fecal odor measuring apparatus 12 includes a sensor unit 17, a supply unit 18, an input unit 19, a communication unit 20, a storage unit 21, and a controller 22.

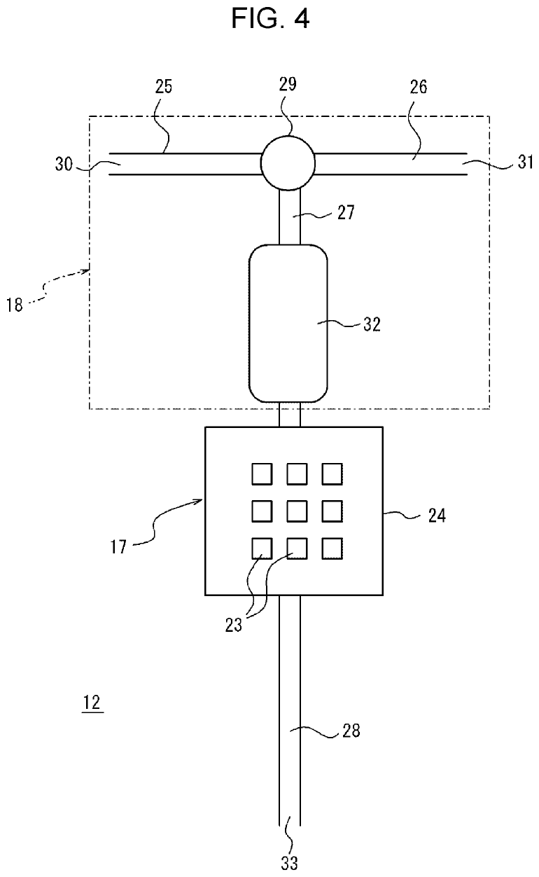

The sensor unit 17 generates and outputs a signal with a signal value in accordance with the concentration of a specific gas. The sensor unit 17 may output the generated signal to the controller 22. As illustrated in FIG. 4, the sensor unit 17 may include a plurality of sensors 23. For example, the sensor unit 17 may include a chamber 24, and the plurality of sensors 23 may be arranged in the chamber 24 to generate signals in response to the same gas. Each of the sensors 23 may have a different sensitivity to the concentration of a specific gas. The specific gas may include, for example, at least one of hydrogen, carbon dioxide, methane, hydrogen sulfide, methyl mercaptan, dimethyl sulfide, carboxylic acid, and amine.

The sensors 23 may be any sensors of the related art, such as semiconductor sensors, contact combustion sensors, and electrochemical sensors. For example, in the case where the sensors 23 are electrochemical sensors, the sensors 23 are each formed of an electrode, an electrolyte, etc. An electrochemical sensor may adjust its sensitivity to a specific gas concentration from a difference between the electrode material and the electrolyte composition. In addition, in the case where the sensors 23 are semiconductor sensors including a metal oxide semiconductor material, the sensitivity to the concentration of a specific gas may be adjusted by appropriately selecting the type of the metal oxide semiconductor material and an impurity to be added.

The supply unit 18 supplies a first gas and a second gas that are different in at least either of obtaining position and obtaining time to the sensor unit 17. For example, in the present embodiment, the supply unit 18 supplies the first gas and the second gas that are different in at least obtaining position to the sensor unit 17.

More specifically, the supply unit 18 includes a first supply passage 25, a second supply passage 26, a third supply passage 27, and an exhaust passage 28.

The first supply passage 25, the second supply passage 26, and the third supply passage 27 are tubes formed of any material such as resin or metal. First ends of the first supply passage 25, the second supply passage 26, and the third supply passage 27 are connected to a three-way valve 29. The first inlet 30 and the second inlet 31, which are second ends of the first supply passage 25 and the second supply passage 26, are provided at different positions in the fecal odor measuring apparatus 12. A second end of the third supply passage 27 is connected to the chamber 24 of the sensor unit 17.

For example, as illustrated in FIG. 2, with the fecal odor measuring apparatus 12 installed in the toilet 14 at a certain position and in a certain posture, the first inlet 30 of the fecal odor measuring apparatus 12 is positioned so as to be exposed inside the toilet bowl 15. In addition, in this state, the second inlet 31 of the fecal odor measuring apparatus 12 is positioned so as to be exposed outside the toilet bowl 15. In the fecal odor measuring apparatus 12 installed in the toilet 14 at such a certain position and in such a certain posture, when a subject defecates, the first gas may be inhaled from the first inlet 30 as a sample gas containing a specific gas to be detected by the sensor unit 17. In addition, the second gas may be inhaled at any time from the second inlet 31 as a purge gas to be detected by the sensor unit 17.

As illustrated in FIG. 4, the third supply passage 26 may be provided with an air supply unit 32. The air supply unit 32 is a pump such as a piezoelectric pump or a motor pump. The air supply unit 32 supplies gases inhaled from the first inlet 30 and the second inlet 31 to the chamber 24. The air supply unit 32 is controlled by the controller 22 to switch the supply of gas on and off.

The three-way valve 29 is capable of switching communication with the third supply passage 26 to either of the first supply passage 24 and the second supply passage 25. The three-way valve 29 performs switching in response to a command from the controller 22. The three-way valve 29 is controlled by the controller 22 to switch communication with the third supply passage 26 to either of the first supply passage 24 and the second supply passage 25.

Each of the first supply passage 25 and the second supply passage 26 may be provided with a storage tank for storing adsorbent. With such a configuration, a gas to be supplied to the sensor unit 17 may be concentrated.

The exhaust passage 28 is a tube formed of any material such as resin or metal. A first end of the exhaust passage 28 is coupled to the chamber 24. The exhaust passage 28 allows a gas supplied to the sensor unit 17 to be exhausted from the sensor unit 17. In the fecal odor measuring apparatus 12, the outlet 33, which is a second end of the exhaust passage 28, is distant from both the first inlet 30 and the second inlet 31. For example, as illustrated in FIG. 2, with the fecal odor measuring apparatus 12 installed in the toilet 14 at a certain position and in a certain posture, the outlet 33 of the fecal odor measuring apparatus 12 is positioned so as to be exposed outside the toilet bowl 15 toward a direction different from the second inlet 31. The exhaust passage 28 may be provided with an air supply unit that supplies a gas in the chamber 24 to the outlet 33.

In FIG. 3, the input unit 19 is, for example, a button. The input unit 19 detects a subject's input indicating an instruction to start fecal odor measurement. In response to detection of the input, the input unit 19 informs the controller 22 that the input has been detected. Alternatively, the input unit 19 may include, for example, a seating sensor. When the seating sensor detects that a subject has seated, the controller 19 may be informed of an instruction to start fecal odor measurement.

The input unit 19 may detect a user input that identifies an individual. In response to pressing of the button, for example, the input unit 19 detects a user input that identifies that a person who has pressed the button is an individual registered for the button. Alternatively, the input unit 19 may include, for example, an image camera, and the input unit 19 may correctly specify the user of the toilet who has been imaged by the image camera and output a signal indicating the specified individual to the controller 22.

The communication unit 20 is capable of communicating with the health condition estimation apparatus 10 and a corresponding terminal apparatus 13. The communication method used for communication between the communication unit 20 and the health condition estimation apparatus 10 may be a wireless communication standard for connecting to a cellular phone network or a wired communication standard. The communication method used for communication between the communication unit 20 and the terminal apparatus 13 may be a short-distance wireless communication standard or a wireless communication standard for connecting to a cellular phone network, or may be a wired communication standard. The short-distance wireless communication standard may include, for example, WiFi (registered trademark), Bluetooth (registered trademark), infrared rays, and NFC (Near Field Communication). The wireless communication standard for connecting to a cellular phone network may include, for example, LTE (Long Term Evolution) or 4G and higher mobile communication systems.

The storage unit 21 includes one or more memories. In the present embodiment, the term “memory” refers to, for example, semiconductor memory, magnetic memory, or optical memory, but these are not the only possible types. Each memory included in the storage unit 21 may function as, for example, a main storage, auxiliary storage, or cache memory. The storage unit 21 may store a formula for calculating the concentration of a specific gas. The storage unit 21 may store any information used for the operation of the fecal odor measuring apparatus 12. The storage unit 21 may store, for example, system programs and application programs.

The formula for calculating the concentration of a gas is calculated in advance by conducting, for example, a multiple regression analysis using a later-described explanatory variable, which is based on the signal value of a signal of each of the sensors 23 when a gas having a known concentration is supplied to the sensor unit 17, and the known concentration. Note that the formula for calculating a gas concentration may be updated by being given from the health condition estimation apparatus 10, as will be described later.

The controller 22 includes one or more processors and memory. The processor(s) may include at least either of a general-purpose processor that is loaded with a specific program and that performs a specific function, and a dedicated processor specialized for specific processing. The dedicated processor may include an application specific integrated circuit (ASIC). The processor(s) may include a programmable logic device (PLD). The PLD may include an FPGA (Field-Programmable Gate Array). The controller 22 may include at least either of SoC (System-on-a-Chip) where one or more processors cooperate, and SiP (Systems-in-a-Package).

The controller 22 controls the supply unit 18 when the controller 22 is informed by the input unit 19 that a subject's input indicating an instruction to start fecal odor measurement has been detected. Alternatively, the controller 22 may control the supply unit 18 when the controller 22 is informed by a seating sensor apparatus, which is another piece of equipment of the fecal odor measuring apparatus 12, via the communication unit 20 that a subject's seating has been detected. In controlling the supply unit 18, the controller 22 allows either of the first gas inhaled from the first inlet 30 and the second gas inhaled from the second inlet 31 to be supplied to the sensor unit 17.

The controller 22 may supply the combination of the first gas and the second gas, which are supplied in sequence, a plurality of times. The number of times this combination is supplied is, for example, four times. Before supplying the combination, the controller 22 may supply the second gas.

The controller 22 may store, as an explanatory variable in the storage unit 21, the signal value of a signal obtained from the sensor unit 17 in each of a plurality of time sections, which are obtained by dividing a period in which the first gas is supplied to the sensor unit 17 into a plurality of time sections. The controller 22 may store, as an explanatory variable in the storage unit 21, the signal value of a signal obtained from the sensor unit 17 in each of a plurality of time sections, which are obtained by dividing a period in which the second gas is supplied to the sensor unit 17 into a plurality of time sections.

The controller 22 may calculate at least either of at least one of the mean, median, and slope of the signal value of a signal in each of the time sections, and the difference in at least one of the mean, median, and slope from a different time section, and store the calculated result as an explanatory variable in the storage unit 21. Furthermore, the controller 22 may calculate the ratio for each of the sensors 23 of at least either of at least one of the mean, median, and slope of the signal value of a signal in each of the time sections, and the difference in at least one of the mean, median, and slope from a different time section, and store the calculated ratio as an explanatory variable in the storage unit 21.

As described above, in the configuration where the combination of the first gas and the second gas is supplied in sequence a plurality of times, the controller 22 may store the above-mentioned explanatory variable in the storage unit 21 for every supply of the combination. Alternatively, as described above, in the configuration where the combination of the first gas and the second gas is supplied in sequence a plurality of times, the controller 22 may store the mean or median of values regarded as explanatory variables for every supply of the combination, as described above, as an explanatory variable in the storage unit 21.

Using the explanatory variable, the controller 22 generates gas information. The gas information is information that includes the concentration of each of specific gases in the present embodiment. The controller 22 calculates the concentration of each of the specific gases by substituting the latest explanatory variable stored in the storage unit 21 into the calculation formula stored in the storage unit 21. For example, the controller 22 calculates a concentration yₙ of a specific gas by using a calculation formula expressed by Formula (1).

[Formula 1]

yₙ=ΣᵢΣⱼΣₖaᵢⱼₖₙ×xᵢⱼₖ+bₙ (1)

In Formula (1), i, j, k, and n are natural numbers. is a number specifying a time section corresponding to the explanatory variable. For example, in the case where a period in which the first gas is supplied to the sensor unit 17 and a period in which the second gas is supplied to the sensor unit 17 are each divided into five, numbers respectively specifying the five time sections in which the first gas is supplied to the sensor unit 17 may be defined as 1 to 5, and numbers respectively specifying the five time sections in which the second gas is supplied to the sensor unit 17 may be defined as 6 to 10. j is a number specifying a sensor 23 corresponding to the explanatory variable. For example, in the configuration where the sensor unit 17 includes three sensors 23, numbers respectively specifying the three sensors 23 may be defined as 1 to 3. k is a number specifying the type of numerical value corresponding to the explanatory variable. For example, numbers respectively specifying, as explanatory variables, the signal value, mean, median, slope, the difference in these values from a different time section, and the ratio of these values for each sensor 23 may be defined as 1 to 16. n is a number associated with the type of a specific gas to be calculated. The explanatory variable xᵢⱼₖ is a numerical value specified by k on the basis of a signal output by a sensor 23 specified by j in a time section specified by i. The coefficient aᵢⱼₖₙ is a coefficient of the explanatory variable xᵢⱼₖ for calculating the concentration of a gas corresponding to n.

In the configuration where the explanatory variable for every supply of the combination is stored in the storage unit 21, the controller 22 may calculate the concentration of a specific gas for every supply of the combination as a temporary concentration using the explanatory variable for every supply of the combination. The controller 22 may allow the mean or median of the temporary concentration of a specific gas, which is calculated for every supply of the combination, to be included as the concentration of the specific gas in the gas information.

The controller 22 activates the communication unit 20 so as to give the gas information including the calculated gas concentration of the specific gas to the health condition estimation apparatus 10. When giving the gas information to the health condition estimation apparatus 10, the controller 22 may give it in association with the identification information of a subject and the identification information of a terminal apparatus 13 corresponding to the subject. The terminal apparatus 13 corresponding to the subject may be, for example, the terminal apparatus 13 registered for an individual detected by the input unit 19. Alternatively, in the case where a subject specified by face recognition is informed by a face recognition apparatus, which is another piece of equipment of the fecal odor measuring apparatus 12, via the communication unit 20, a terminal apparatus 13 corresponding to the subject may be a terminal apparatus 13 registered for the subject. When giving the gas information to the health condition estimation apparatus 10, the controller 22 may give it in association with the time at which the gas information was generated.

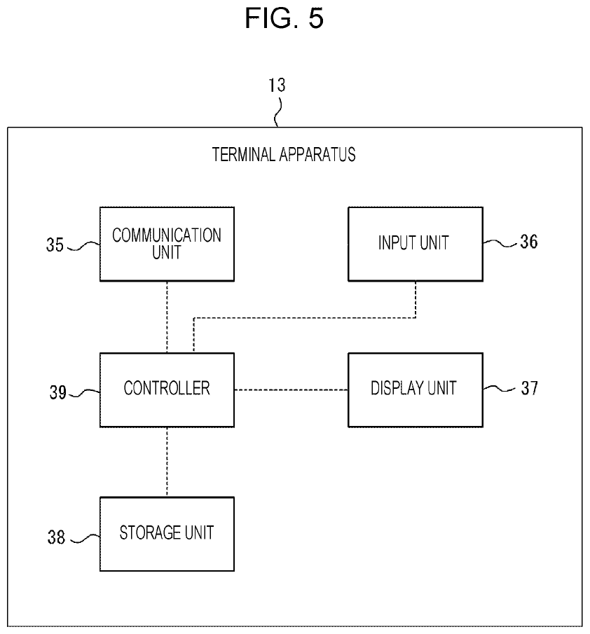

Each terminal apparatus 13 is general electronic equipment such as a smart phone or a PC (Personal Computer), or dedicated electronic equipment. As illustrated in FIG. 5, the terminal apparatus 13 includes a communication unit 35, an input unit 36, a display unit 37, a storage unit 38, and a controller 39.

The communication unit 35 is capable of communicating with the health condition estimation apparatus 10 and a corresponding fecal odor measuring apparatus 12. The communication method used for communication between the communication unit 35 and the health condition estimation apparatus 10 may be a wireless communication standard for connecting to a cellular phone network or a wired communication standard. The communication method used for communication between the communication unit 35 and the fecal odor measuring apparatus 12 may be a short-distance wireless communication standard or a wireless communication standard for connecting to a cellular phone network, or may be a wired communication standard. The communication method used for communication between the communication unit 35 and the health condition estimation apparatus 10 may be a communication standard such as LPWA (Low Power Wide Area) or LPWAN (Low Power Wide Area Network).

The input unit 36 includes one or more input interfaces that detect a user input. The input interface(s) includes, for example, a physical key, an electrostatic key, and a touchscreen integrally provided with the display unit 37. The input unit 36 is capable of detecting an input of a subject's physical information, a subject's food intake information, and a subject's intestinal bacterial status. The input unit 36 gives the input subject's physical information, subject's food intake information, and subject's intestinal bacterial status to the controller 39.

The subject's physical information may include at least one of the subject's sex, age, height, weight, and body fat percentage. The subject's food intake information may include at least one of foods ingested by the subject, nutrients contained in the ingested foods, the time of ingestion, and the frequency of ingestion. The nutrients may include at least one of dietary fiber, starch, oligosaccharides, carbohydrate digestive enzyme inhibitors, and proteins. The subject's intestinal bacterial status may include the number of the subject's specific intestinal bacteria, and may further include the pH of the feces used for inspecting the number of the specific intestinal bacteria. The specific intestinal bacteria may include at least one of bifidobacterium, lactobacillus, clostridium, bacteroides, prevotella, ruminococcus, and esquericia. The number of the subject's specific intestinal bacteria may be measured by an inspection of the subject's feces by an inspection institution or the like.

The display unit 37 is, for example, any display of the related art. The display unit 37 may display health information. The health information is information indicating an intestinal condition regarding a subject's health condition, and may include, for example, at least one of the number of bacteria by type of intestinal bacteria, the ratio of bacterial categories that classify each type of intestinal bacteria, and the health condition and health information based on the intestinal bacterial status. Although the health information in the present embodiment describes the intestinal bacterial status in particular among parts of the digestive tract (oral cavity, pharynx, esophagus, stomach, intestines, etc.), this is not the only possible information, and may take into consideration and include information on residential bacteria in other parts of the digestive tract. In addition, the health information may take into consideration and include, for example, in order to take into consideration the relationship between the brain and intestines, neurotransmitters in the brain, their raw materials and hormones, etc., as in the case of the above-mentioned information indicating the intestinal condition.

The storage unit 38 includes one or more memories. In the present embodiment, the term “memory” refers to, for example, semiconductor memory, magnetic memory, or optical memory, but these are not the only possible types. Each memory included in the storage unit 38 may function as, for example, a main storage, auxiliary storage, or cache memory. The storage unit 38 may store any information used for the operation of the terminal apparatus 13. The storage unit 38 may store, for example, system programs and application programs.

The controller 39 includes one or more processors and memory. The processor(s) may include at least either of a general-purpose processor that is loaded with a specific program and that performs a specific function, and dedicated processor specialized for specific processing. The dedicated processor may include an ASIC. The processor(s) may include a PLD. The PLD may include an FPGA. The controller 39 may include at least either of SoC where one or more processors cooperate, and SiP.

Having obtained at least one of the subject's physical information and the subject's food intake information from the input unit 36, the controller 39 may store the obtained information in the storage unit 38. The controller 39 may give information indicating at least one of the latest physical information and food intake information stored in the storage unit 38 indirectly via the fecal odor measuring apparatus 12 or directly to the health condition estimation apparatus 10. When giving the information, the controller 39 may give it in association with the identification information of the subject. When giving the information, the controller 39 may give it in association with the time at which the information was obtained by the terminal apparatus 13.

As will be described later, on receipt of health information indirectly via the fecal odor measuring apparatus 12 or directly from the health condition estimation apparatus 10, the controller 39 may display the health information on the display unit 37.

In the case where the subject's intestinal bacterial status is obtained from the input unit 36, the controller 39 may store the intestinal bacterial status in the storage unit 38. The controller 39 may drive the communication unit 35 so as to give the intestinal bacterial status stored in the storage unit 38 indirectly via the fecal odor measuring apparatus 12 or directly to the health condition estimation apparatus 10. When giving the intestinal bacterial status, the controller 39 may give it in association with the identification information of the subject, the identification information of the terminal apparatus 13, and the time at which the intestinal bacterial status was obtained.

When giving the intestinal bacterial status to the health condition estimation apparatus 10, the controller 39 may obtain certain benefits from the health condition estimation apparatus 10. The certain benefits may be, for example, making it free of charge or discounting the fee for the right to use services provided by a service provider that uses the health condition estimation apparatus 10.

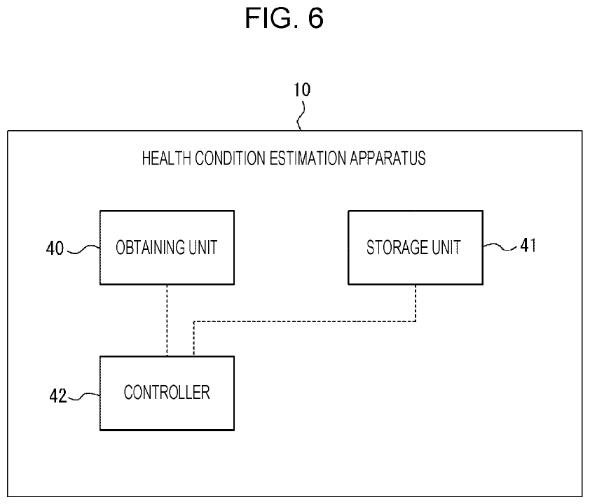

As illustrated in FIG. 6, the health condition estimation apparatus 10 includes an obtaining unit 40, a storage unit 41, and a controller 42. The health condition estimation apparatus 10 is, for example, a server.

The obtaining unit 40 includes, for example, a communication module capable of communicating with the fecal odor measuring apparatuses 12 and the terminal apparatuses 13. The obtaining unit 40 includes, for example, a communication module that connects to a network. The obtaining unit 40 obtains gas information based on a signal output by the sensor unit 17 of each of the fecal odor measuring apparatuses 12. The obtaining unit 40 may obtain a subject's physical information and food intake information.

The storage unit 41 includes one or more memories. In the present embodiment, the term “memory” refers to, for example, semiconductor memory, magnetic memory, or optical memory, but these are not the only possible types. The storage unit 41 stores a database including numerous sets of each subject's physical information, food intake information, intestinal bacterial status, and gas information that are associated with one another. The storage unit 41 stores an estimation formula for generating health information.

The estimation formula for generating health information is calculated in advance by preliminarily measuring the concentration of gas in the fecal odors of a number of subjects using high-precision sensors, linking the measurement results to the results of measuring the subjects' health conditions, and conducting, for example, a multiple regression analysis. Note that the estimation formula for generating health information may be updated using the intestinal bacterial status, such as gas information obtained from each of the numerous fecal odor measuring apparatuses 12 and terminal apparatuses 13, as will be described later.

The controller 42 includes one or more processors and memory. The processor(s) may include at least either of a general-purpose processor that is loaded with a specific program and that performs a specific function, and a dedicated processor specialized for specific processing. The dedicated processor may include an ASIC. The processor(s) may include a PLD. The PLD may include an FPGA. The controller 42 may include at least either of SoC where one or more processors cooperate, and SiP.

Having obtained gas information via the obtaining unit 40, the controller 42 stores the gas information as an explanatory variable in the storage unit 41. By substituting the concentration of a specific gas, which is newly stored in the storage unit 41, into the estimation formula stored in the storage unit 41, the controller 42 calculates the number of bacteria by type of intestinal bacteria or the ratio of bacterial categories that classify each type of intestinal bacteria as health information. The controller 42 may calculate a response variable zₘ by using, for example, an estimation formula expressed by Formula (2).

[Formula 2]

zₘ=Σₗcₗₘ×yₗ+dₘ (2)

In Formula (2), 1 and m are natural numbers. l is a number associated with the type of a specific gas. m is a number specifying the number of bacteria or the bacterial category, which corresponds to the to-be-calculated response variable. yₗ is the concentration of a specific gas of a type associated with l. cₗₘ is the coefficient of the explanatory variable yₗ for calculating the number of bacteria or the ratio of bacterial categories, which corresponds to m. cₗₘ for the type of a specific gas varies according to the type of another gas used in Formula (2). In other words, the coefficient cₗᵢₘ for the type l1 of a specific gas varies depending on the presence or absence of the term cₗ₂ₘ×yₗ₂ of the concentration yₗ₂ of a gas of another type in Formula (2). Therefore, the response variable may be calculated by changing the coefficient cₗₘ used for the calculated gas concentration in accordance with the type of gas whose concentration is not calculated.

The intestinal bacteria may include, for example, those selected from bifidobacterium, lactobacillus, clostridium, bacteroides, prevotella, ruminococcus, and esquericia. The bacterial categories may include, for example, those selected from good bacteria, bad bacteria, opportunistic bacteria, immune-associated bacteria, fat bacteria, and lean bacteria.

The controller 42 stores the calculated health information in the storage unit 41 in association with the identification information of a subject and the identification information of a terminal apparatus 13 that are associated with gas information used for the calculation. The controller 42 drives the obtaining unit 40 so as to give the health information stored in the storage unit 41 to the terminal apparatus 13 on the basis of the identification information of the terminal apparatus 13 associated with the health information.

Having obtained at least one of the subject's physical information and food intake information via the obtaining unit 40, the controller 42 may store the obtained information as an explanatory variable in the storage unit 41. To generate health information, the controller 42 may use at least either of the subject's physical information and the subject's food intake information whose detection time is nearest to the generation time of gas information. More specifically, the controller 42 may calculate the number of bacteria or the ratio of bacterial categories using the subject's age, height, weight, and body fat percentage in addition to the explanatory variable in Formula (2). In addition, the controller 42 may calculate the number of bacteria or the ratio of bacterial categories using the subject's sex, foods ingested by the subject, and nutrients contained in the ingested foods, which are classified into one or zero, in addition to the explanatory variable in Formula (2). Alternatively, the controller 42 may change the coefficients of the explanatory variable according to the subject's sex, foods ingested by the subject, and nutrients contained in ingested foods. In the case of adding at least one of the subject's age, height, weight, body fat percentage, the subject's sex, foods ingested by the subject, and nutrients contained in the ingested foods to the explanatory variable, the controller 42 may change their coefficients in the estimation formula.

On the basis of the calculated number of bacteria or ratio of bacterial categories, the controller 42 may further generate a health condition based on the intestinal bacterial status. For example, the controller 42 may calculate, as a health condition, the intestinal health score based on the balance of the numbers of good bacteria, bad bacteria, and opportunistic bacteria, the physical condition score based on the number of immune-related bacteria, and the obesity tendency score based on the balance of the numbers of fat bacteria and lean bacteria.

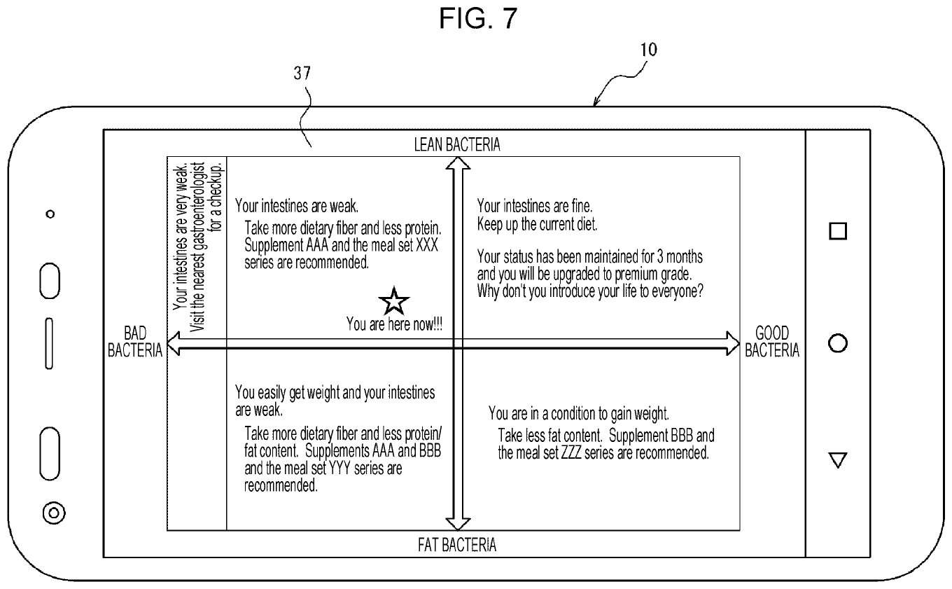

On the basis of the calculated number of bacteria or ratio of bacterial categories, the controller 42 may further generate a health advice based on the intestinal bacterial status as health information. The controller 42 may generate, for example, as a health advice, as illustrated in FIG. 7, an image indicating the subject's position in a matrix with the balance of the proportions of good bacteria and bad bacteria on the horizontal axis and the balance of the proportions of fat bacteria and lean bacteria on the vertical axis. In the matrix, for example, a health advice is assigned by horizontal axis and vertical axis. FIG. 7 illustrates one mode of display of the display unit 37. On the display unit 37, diagrams, graphs, etc. may be displayed as appropriate in accordance with display items.

In the case where the intestinal bacterial status is obtained from one of the terminal apparatuses 13 via the obtaining unit 40, the controller 42 stores the intestinal bacterial status in the storage unit 41. When storing the intestinal bacterial status, the controller 42 associates the identification information of a subject associated with the intestinal bacterial status, and an explanatory variable used for generating the health information, to which the same identification information is associated.

Under a certain condition, the controller 42 updates the estimation formula stored in the storage unit 41 on the basis of an explanation variable and a response variable that have been trained in advance including machine learning. The certain condition includes, for example, a certain cycle, a certain time, and when the number included in the intestinal bacterial status obtained after the update exceeds a threshold. Using the database stored in the storage unit 41, the controller 42 conducts, for example, a multiple regression analysis to update the estimation formula. Note that the controller 42 may conduct, in addition to a multiple regression analysis, a cluster analysis or a principal component analysis. Specifically, the controller 42 updates the coefficients cₗₘ and dₘ in Formula (2) on the basis of the gas concentration yₗ in the database and the response variable zₘ in Formula (2). The controller 42 stores the updated estimation formula in the storage unit 41, which may be used for generating health information in the future.

Depending on how much the sensors 23 of each fecal odor measuring apparatus 12 are deteriorated, the controller 42 may drive the obtaining unit 40 so as to give the changed coefficients aᵢⱼₖₙ and bₙ in Formula (1) to the fecal odor measuring apparatus 12.

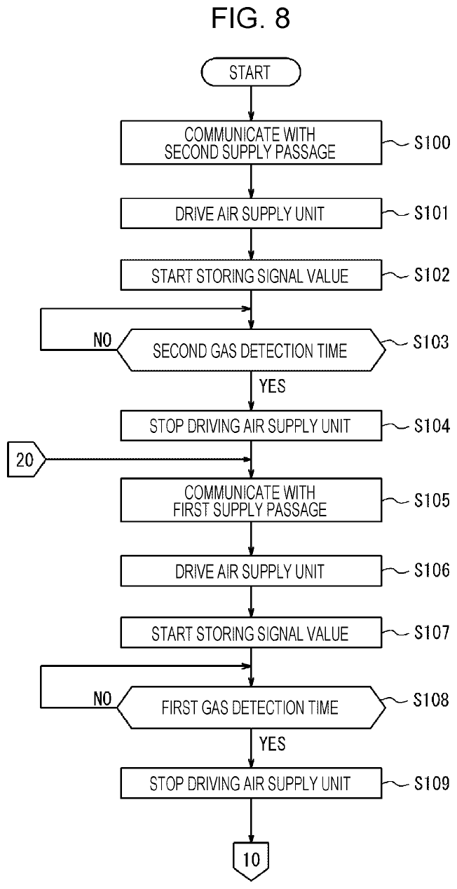

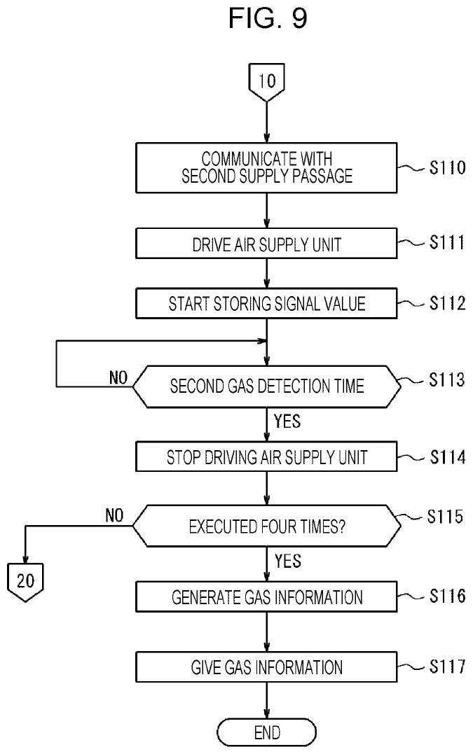

Next, a gas information generating process, which is executed by the controller 22 of the fecal odor measuring apparatus 12 in the present embodiment, will be described using the flowcharts in FIGS. 8 and 9. The gas information generating process starts when the input unit 19 detects an input indicating an instruction to start fecal odor measurement.

In step S100, the controller 22 drives the three-way valve 29 so that the third supply passage 27 will be communicated with the second supply passage 26. After the three-way valve 29 is driven, the process proceeds to step S101.

In step S101, the controller 22 drives the air supply unit 32 to supply the second gas from the second inlet 31 to the sensor unit 17. After the air supply unit 32 is driven, the process proceeds to step S102.

In step S102, the controller 21 starts storing signals that are continuously obtained from the sensor unit 17 in the storage unit 20. After the storage is started, the process proceeds to step S103. In a configuration where the fecal odor measuring apparatus 12 calculates an explanatory variable other than signal values based on the signal values of signals output by the sensor unit 17, the controller 22 calculates and stores the explanatory variable in the storage unit 20.

In step S103, the controller 22 determines whether a second gas detection time determined as a time of supplying the second gas from the second inlet 31 to the sensor unit 17 has elapsed from the time at which the driving of the air supply unit 32 is started in step S101. In the case where the second gas detection time has not elapsed, step S103 is repeated. In the case where the second gas detection time has elapsed, the process proceeds to step S104.

In step S104, the controller 22 stops driving the air supply unit 32. After the stop, the process proceeds to step S105.

In step S105, the controller 22 drives the three-way valve 29 so that the third supply passage 27 will be communicated with the first supply passage 25. After the three-way valve 29 is driven, the process proceeds to step S106.

In step S106, the controller 22 drives the air supply unit 32 to supply the first gas from the first inlet 30 to the sensor unit 17. After the air supply unit 32 is driven, the process proceeds to step S107.

In step S107, the controller 22 starts storing signals that are continuously obtained from the sensor unit 17 in the storage unit 20. After the storage is started, the process proceeds to step S108. In a configuration where the fecal odor measuring apparatus 12 calculates an explanatory variable other than signal values based on the signal values of signals output by the sensor unit 17, the controller 22 calculates and stores the explanatory variable in the storage unit 20.

In step S108, the controller 22 determines whether a first gas detection time determined as a time of supplying the first gas from the first inlet 30 to the sensor unit 17 has elapsed from the time at which the driving of the air supply unit 32 is started in step S106. In the case where the first gas detection time has not elapsed, step S108 is repeated. In the case where the first gas detection time has elapsed, the process proceeds to step S109.

In step S109, the controller 22 stops driving the air supply unit 32. After the driving is stopped, the process proceeds to step S110.

In step S110, the controller 22 drives the three-way valve 29 so that the third supply passage 27 will be communicated with the second supply passage 26. After the three-way valve 29 is driven, the process proceeds to step S111.

In step S111, the controller 22 drives the air supply unit 32 to supply the second gas from the second inlet 31 to the sensor unit 17. After the air supply unit 32 is driven, the process proceeds to step S112.

In step S112, the controller 21 starts storing signals that are continuously obtained from the sensor unit 17 in the storage unit 20. After the storage is started, the process proceeds to step S113. In a configuration where the fecal odor measuring apparatus 12 calculates an explanatory variable other than signal values based on the signal values of signals output by the sensor unit 17, the controller 22 calculates and stores the explanatory variable in the storage unit 20.

In step S113, the controller 22 determines whether a second gas detection time determined as a time of supplying the second gas from the second inlet 31 to the sensor unit 17 has elapsed from the time at which the driving of the air supply unit 32 is started in step S111. In the case where the second gas detection time has not elapsed, step S113 is repeated. In the case where the second gas detection time has elapsed, the process proceeds to step S114.

In step S114, the controller 22 stops driving the air supply unit 32. After the stop, the process proceeds to step S115.

In step S115, the controller 22 determines whether the control from steps S105 to S114 has been executed four times after the start of the information generating process. In the case where the control has not been executed four times, the process returns to step S105. In the case where the control has been executed four times, the process proceeds to step S116.

In step S116, the controller 22 calculates the concentrations of specific gases using the explanatory variable stored in the storage unit 21 on the basis of, among signal values stored in the storage unit 20, signal values received during the control in steps S105 to S113. The controller 22 generates gas information including all the concentrations of specific gases that should be calculated. After the generation, the process proceeds to step S117.

In step S117, the controller 22 gives the gas information generated in step S116 to the health condition estimation apparatus 10. After the gas information is given, the gas concentration generating process ends.

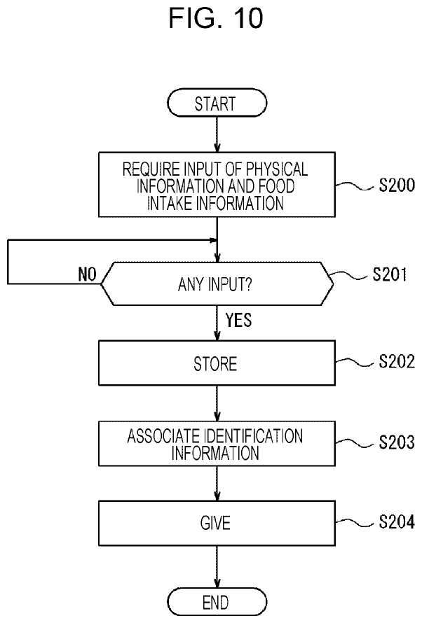

Next, a physical information etc. giving process, which is executed by the controller 39 of the terminal apparatus 13 in the present embodiment, will be described using the flowchart in FIG. 10. The physical information etc. giving process starts in the case where the input unit 36 detects an input requiring at least either of a subject's physical information and food intake information to be provided.

In step S200, the controller 39 requires an input of physical information and food intake information by, for example, displaying an image on the display unit 37. After an input is required, the process proceeds to step S201.

In step S201, the controller 39 determines whether there is an input of at least one of physical information and food intake information from the subject. An input of physical information and food intake information mentioned above may be an input of selection from items of physical information and food intake information that are input in the past and stored in the storage unit 38. In the case where the information is not input, the process returns to step S201. In the case where the information is input, the process proceeds to step S202.

In step S202, the controller 39 stores at least one of the physical information and the food intake information whose input has been confirmed in step S201 in the storage unit 28. After the storage, the process proceeds to step S203.

In step S203, the controller 39 associates at least one of the physical information and the food intake information that has been stored in the storage unit 38 in step S202 with the identification information of the subject and the identification information of the terminal apparatus 13. After the association, the process proceeds to step S204.

In step S204, the controller 39 gives at least one of the physical information and the food intake information that has been associated with the identification information in step S203 to the health condition estimation apparatus 10. After the information is given, the physical information etc. giving process ends.

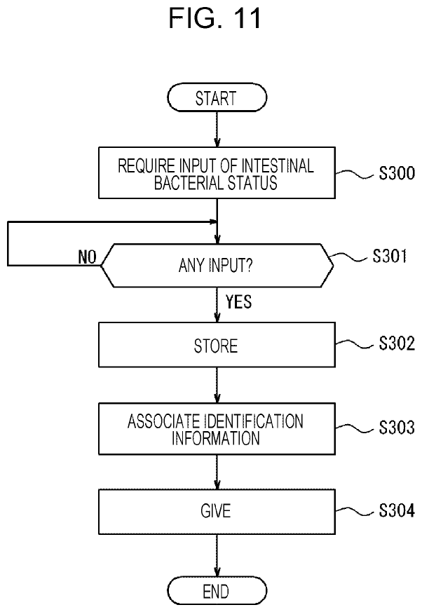

Next, an intestinal status giving process, which is executed by the controller 39 of the terminal apparatus 13 in the present embodiment, will be described using the flowchart in FIG. 11. The intestinal status giving process starts in the case where the input unit 36 detects an input requiring a subject's intestinal bacterial status to be provided.

In step S300, the controller 39 requires an input of the intestinal bacterial status by, for example, displaying an image on the display unit 37. After an input is required, the process proceeds to step S301.

In step S301, the controller 39 determines whether there is an input of the intestinal bacterial status from the subject. In the case where there is no input, the process returns to step S301. In the case where there is an input, the process proceeds to step S302.

In step S302, the controller 39 stores the intestinal bacterial status whose input has been confirmed in step S301 in the storage unit 28. After the storage, the process proceeds to step S303.

In step S303, the controller 39 associates the intestinal bacterial status stored in the storage unit 38 in step S302 with the identification information of the subject and the identification information of the terminal apparatus 13. After the association, the process proceeds to step S304.

In step S304, the controller 39 gives the intestinal bacterial status, which has been associated with the identification information in step S303, to the health condition estimation apparatus 10. After the status is given, the intestinal status giving process ends.

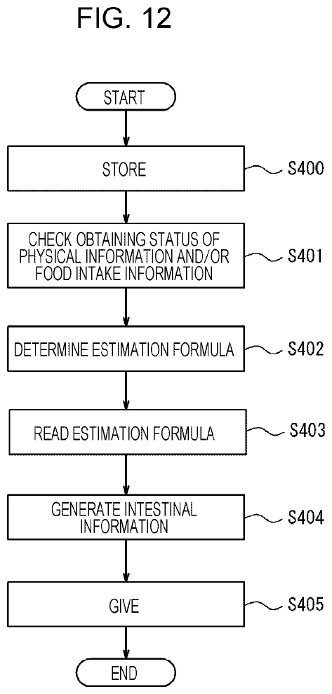

Next, a health information generating process, which is executed by the controller 42 of the health condition estimation apparatus 10 in the present embodiment, will be described using the flowchart in FIG. 12. The health information generating process starts in the case where gas information is obtained.

In step S400, the controller 39 stores the obtained gas information in the storage unit 38. After the storage, the process proceeds to step S401.

In step S401, the controller 39 checks the obtaining status of at least one of physical information and food intake information from a subject that corresponds to the identification information of a subject associated with the gas information. After the checking, the process proceeds to step S402.

In step S402, the controller 39 determines the estimation formula to use on the basis of the type of item of at least one of the physical information and the food intake information that has been checked in step S401. That is, the controller 39 determines each explanatory variable coefficient determined by an input item, as described above. After the determination, the process proceeds to step S403.

In step S403, the controller 39 reads the estimation formula determined in step S401 from the storage unit 38. After the estimation formula is read, the process proceeds to step S404.

In step S404, the controller 39 generates health information (intestinal information in the example in FIG. 12) using the gas information stored in the storage unit 38 in step S400 and the physical information and food intake information checked in step S401. The controller 39 stores the generated health information in the storage unit 38 in association with the identification information of the subject and the identification information of the terminal apparatus 13. After the generation, the process proceeds to step S405.

In step S405, the controller 39 gives the health information generated in step S404 to the terminal apparatus 13 on the basis of the identification information of the terminal apparatus 13 associated with the gas information. After the health information is given, the health information generating process ends.

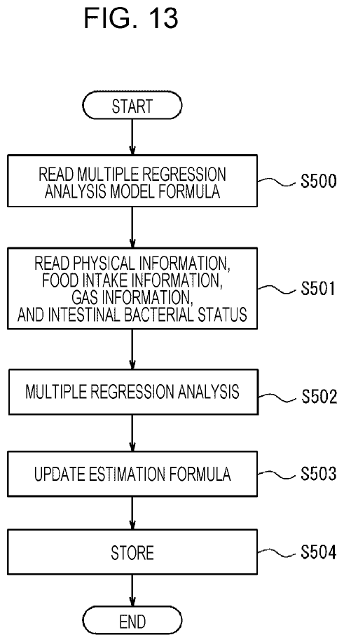

Next, an estimation formula updating process, which is executed by the controller 42 of the health condition estimation apparatus 13 in the present embodiment, will be described using the flowchart in FIG. 13. The estimation formula updating process starts in the case where the above-mentioned certain condition is satisfied.

In step S500, the controller 42 reads a model formula for a multiple regression analysis from the storage unit 41. After the model formula is read, the process proceeds to step S501. The above-mentioned model formula is a formula for calculating the intestinal bacterial status from at least the concentration of a specific gas and, if available, at least one of physical information and food intake information, and the coefficients of the current estimation formula may be used as the initial values of coefficients.

In step S501, the controller 42 reads a plurality of sets of physical information, food intake information, gas information, and intestinal bacterial status that are associated with the same subject identification information from the storage unit 41. After the sets of information are read, the process proceeds to step S502.

In step S502, the controller 42 conducts a multiple regression analysis on the model formula read in step S500 using the gas concentration included in the gas information read in step S501 and the plurality of sets of physical information, food intake information, and intestinal bacterial status read in step S601. That is, the controller 42 calculates a regression coefficient that minimizes, as a cost function, the mean square value of the difference between the intestinal bacterial status calculated by substituting, for example, a specific gas concentration, physical information, and food intake information into the model formula and the intestinal bacterial status read from the storage unit 41. After the calculation, the process proceeds to step S502.

In step S503, the controller 42 updates the estimation formula using the regression coefficient calculated in step S502. After the update, the process proceeds to step S504.

In step S504, the controller 42 stores the estimation formula updated in step S503 in the storage unit 41. After the storage, the estimation formula updating process ends.

In the health condition estimation apparatus 10 of the present embodiment with the foregoing configuration, health information is generated on the basis of gas information including signals output by the sensor unit 17 in a period in which the first gas is supplied to the sensor unit 17 and signals output by the sensor unit 17 in a period in which the second gas is supplied to the sensor unit 17. The health information is, as described above, information indicating the intestinal condition regarding a subject's health condition, and is generated on the basis of the intestinal bacterial status. The intestinal bacterial status may be estimated on the basis of odor emitted from the subject's feces, which is, in other words, the concentrations of various gases. Therefore, a simple estimation of health information may be performed by estimating the intestinal bacterial status on the basis of the result of detecting the concentrations of various gases contained in feces. Because the health condition estimation apparatus 10 with the foregoing configuration uses not only the first gas, which may be a sample gas, but also the second gas, which may be a purge gas, for gas information, the health condition estimation apparatus 10 may obtain signals for estimating the concentrations of the specific gases and consequently may estimate the intestinal bacterial status. In contrast, because the health condition estimation apparatus 10 estimates the intestinal bacterial status by specific signal processing without having a special configuration compared with a configuration of the related art, the health condition may be estimated with a simple configuration.

In the health condition estimation apparatus 10 of the present embodiment, signals output by the sensor unit 17 in a period in which the first gas is supplied to the sensor unit 17 are signals output by the sensor unit 17 in the individual time sections, which are obtained by dividing the period in which the first gas is supplied to the sensor unit 17 into a plurality of time sections. In addition, in the health condition estimation apparatus 10, signals output by the sensor unit 17 in a period in which the second gas is supplied to the sensor unit 17 are signals output by the sensor unit 17 in the individual time sections, which are obtained by dividing the period in which the second gas is supplied to the sensor unit 17 into a plurality of time sections. In general, a signal value itself in each of sections obtained by dividing a period from the start of inhalation of a sample gas or a purge gas until the signal value reaches a steady value, and a slope, mean, median, etc. based on the signal value change according to the gas concentration. Therefore, compared with a configuration where signals of the sensor unit 17 that have reached a steady value are used as they are, the health condition estimation apparatus 10 with the foregoing configuration may improve the accuracy of calculating the concentration of a specific gas and consequently may improve the accuracy of estimating the intestinal bacterial status generated from the concentration of the specific gas.

In addition, the sensor unit 17 includes the plurality of sensors 23 in the health condition estimation apparatus 10 of the present embodiment. With such a configuration, compared with a configuration with one sensor 23, the health condition estimation apparatus 10 may improve the accuracy of calculating the concentration of a specific gas and consequently may further improve the accuracy of estimating the intestinal bacterial status.

In the health condition estimation apparatus 10 of the present embodiment, a specific gas to be detected by the sensor unit 17 includes at least one of hydrogen, carbon dioxide, methane, hydrogen sulfide, methyl mercaptan, dimethyl sulfide, carboxylic acid, and amine. In general, gases produced by the intestinal bacteria are different according to the type of intestinal bacteria, and each type contains hydrogen, carbon dioxide, methane, hydrogen sulfide, methyl mercaptan, dimethyl sulfide, carboxylic acid, amine, and the like. Therefore, with the foregoing configuration, the health condition estimation apparatus 10 may further improve the accuracy of estimating the intestinal bacterial status by calculating the concentrations of these specific gases.

In addition, the health condition estimation apparatus 10 of the present embodiment generates health information additionally on the basis of a subject's physical information. The intestinal bacterial status varies depending not only on the concentration of a specific gas, but also on the subject's physical characteristics such as age, height, weight, and body fat percentage. Therefore, with the foregoing configuration, the health condition estimation apparatus 10 may further improve the accuracy of estimating the intestinal bacterial status.

In addition, the health condition estimation apparatus 10 of the present embodiment generates health information additionally on the basis of a subject's food intake information. The intestinal bacterial status varies not only on the basis of the concentration of a specific gas, but also on foods ingested by the subject and nutrients delivered into the intestines such as nutrients contained in the ingested foods. Therefore, with the foregoing configuration, the health condition estimation apparatus 10 may further improve the accuracy of estimating the intestinal bacterial status.

In addition, in the case where a subject's intestinal bacterial status is obtained, the health condition estimation apparatus 10 of the present embodiment associates the intestinal bacterial status with information used for generating health information, such as gas information, via the identification information of the subject. With such a configuration, the health condition estimation apparatus 10 may use the intestinal bacterial status, which is the actually measured number of bacteria in the intestines, as information for conducting a regression analysis on the estimation formula, along with information for estimating health information.

Although the present invention has been described on the basis of the drawings and the embodiment, it shall be noted that various modifications and changes may be easily made by those skilled in the art on the basis of the present disclosure. Therefore, it shall be noted that these modifications and changes fall within the scope of the present invention.

For example, although the health condition estimation apparatus 10 is configured to obtain the concentration of a specific gas, which is generated by the fecal odor measuring apparatus 12, as gas information in the present embodiment, the health condition estimation apparatus 10 may be configured to obtain an explanatory variable for calculating the concentration of a gas as gas information on the basis of a signal detected by the sensor unit 17 of the fecal odor measuring apparatus 12. In this configuration, the health condition estimation apparatus 10 may calculate the concentration of a specific gas on the basis of the gas information. Alternatively, the health condition estimation apparatus 10 may obtain a signal detected by the sensor unit 17 of the fecal odor measuring apparatus 12 and calculate an explanatory variable for calculating the concentration of a gas. Furthermore, in this configuration, the health condition estimation apparatus 10 may be configured to generate health information directly from the explanatory variable, without calculating the concentration of a specific gas from the explanatory variable, using an estimation formula for generating health information.

In addition, although the supply unit 18 of the fecal odor measuring apparatus 12 is configured to obtain the first gas and the second gas at different obtaining positions in the specific configuration of the present embodiment, the first gas and the second gas may be measured at the same position but at different measurement times. For example, in a configuration where the second inlet 31 of the present embodiment is not provided, the same advantageous effects as those of the present embodiment may be achieved by using a gas inhaled from the first inlet 30 with feces in the toilet bowl 15 as the first gas, and a gas inhaled from the first inlet 30 after the feces are evacuated from the toilet bowl 15 as the second gas.

In addition, although the fecal odor measuring apparatus 12 is configured to start supplying the first gas and the second gas to the sensor unit 17 in response to detection of an input to the input unit 19 of the fecal odor measuring apparatus 12 in the present embodiment, a trigger for starting the supply is not limited to detection of an input to the input unit 19. For example, the fecal odor measuring apparatus 12 may be configured to start the supply on the basis of the fact that a subject has seated, for example, by using a human-sensitive sensor such as an infrared sensor or a pressure-sensitive sensor.

In addition, in the present embodiment, the health condition estimation apparatus 10 obtains gas information from the fecal odor measuring apparatus 12, obtains the subject's physical information, the subject's food intake information, and the subject's intestinal bacterial status from the terminal apparatus 13, generates health information on the basis of the gas information, and gives intestinal information to the terminal apparatus 13. However, at least part of processing performed by the health condition estimation apparatus 10 may be performed by the fecal odor measuring apparatus 12 or the terminal apparatus 13. Furthermore, the fecal odor measuring apparatus 12 and the terminal apparatus 13 may be an integrated apparatus.

In a configuration where generation of health information is performed by the fecal odor measuring apparatus 12 or the terminal apparatus 13, the fecal odor measuring apparatus 12 or the terminal apparatus 13 may give the calculated health information to the health condition estimation apparatus 10, which serves as an analysis apparatus, in association with the identification information of a subject associated with gas information used for generating the health information. In this configuration, the fecal odor measuring apparatus 12 or the terminal apparatus 13 may obtain the estimation formula updated by the health condition estimation apparatus 10 and use it for generating health information in the future.

Although the fecal odor measuring apparatus 12 allows a signal obtained from the sensor unit 17 in each of time sections, which are obtained by dividing a period in which the first gas is supplied to the sensor unit 17 into a plurality of time sections, to be included in gas information in the present embodiment, this is not the only possible configuration. The same applies to a period in which the second gas is supplied.

For example, the fecal odor measuring apparatus 12 may perform the Fourier expansion of the waveform of a signal obtained from the sensor unit 17 in a period in which the eleventh gas is supplied to the sensor unit 17, separate it into a polynomial, and allow each coefficient in the polynomial to be included in gas information. In such a configuration, the health condition estimation apparatus 10 may calculate the gas concentration or health information (response variable zₘ) on the basis of an estimation formula in which each coefficient of the polynomial expansion serves as an explanatory variable.

In addition, for example, the fecal odor measuring apparatus 12 may convert, by performing a Fourier transform, the waveform of a signal obtained from the sensor unit 17 in a period in which the eleventh gas is supplied to the sensor unit 17 from a function of time to a function of frequency, and allow the function of frequency to be included in gas information. In such a configuration, the health condition estimation apparatus 10 may calculate the gas concentration or health information (response variable zₘ) on the basis of an estimation formula in which the mean, median, and slope in each frequency section, and the ratio of the mean, median, and slope serve as explanatory variables.

Unless otherwise specified, networks used here include the Internet, an ad hoc network, LAN (Local Area Network), WAN (Wide Area Network), MAN (Metropolitan Area Network), cellular network, WWAN (Wireless Wide Area Network), WPAN (Wireless Personal Area Network), PSTN (Public Switched Telephone Network), terrestrial wireless network, other networks, or a combination thereof. The components of a wireless network include, for example, an access point (such as Wi-Fi access point), femtocell, and the like. Furthermore, a wireless communication device is capable of connecting to a wireless network using Wi-Fi, Bluetooth, cellular communication technologies (such as CDMA (Code Division Multiple Access), TDMA (Time Division Multiple Access), FDMA (Frequency Division Multiple Access), OFDMA (Orthogonal Frequency Division Multiple Access), or SC-FDMA (Single-Carrier Frequency Division Multiple Access)), or other wireless technologies and/or technical standards. A network may adopt one or more technologies, and these technologies include, for example, UTMS (Universal Mobile Telecommunications System), LTE (Long Term Evolution), EV-DO (Evolution-Data Optimized or Evolution-Data Only), GSM (Global System for Mobile communications), WiMAX (Worldwide Interoperability for Microwave Access), CDMA-2000 (Code Division Multiple Access-2000) or TD-SCDMA (Time Division Synchronous Code Division Multiple Access).

It shall be noted here that a system is disclosed as having various modules and/or units that perform specific functions, and these modules and units are schematically indicated to briefly describe their functionality, and do not necessarily indicate specific pieces of hardware and/or software. In that sense, it is only necessary that these modules, units, and other components be pieces of hardware and/or software implemented to substantially execute specific functions described here. Various functions of different components may be any combination of or separated pieces of hardware and/or software, which may be used separately or in any combination. In addition, input/output or I/O devices, or user interfaces, including but not limited to keyboards, displays, touchscreens, and pointing devices, may be directly connected to the system or through intervening I/O controllers. As has been described above, various aspects of the contents of the present disclosure may be implemented in many different modes, and all of these modes are included within the scope of the contents of the present disclosure.

### REFERENCE SIGNS LIST

10 health condition estimation apparatus
11 health condition estimation system
12 fecal odor measuring apparatus
13 terminal apparatus
14 toilet
15 toilet bowl
16 toilet seat
17 sensor unit
18 supply unit
19 input unit
20 communication unit
21 storage unit
22 controller
23 sensors
24 chamber
25 first supply passage
26 second supply passage
27 third supply passage
28 exhaust passage
29 three-way valve
30 first inlet
31 second inlet
32 air supply unit
33 outlet
35 communication unit
36 input unit
37 display unit
38 storage unit
39 controller
40 obtaining unit
41 storage unit
42 controller

1. A health condition estimation apparatus comprising:
an obtaining unit configured to obtain, from a sensor unit that outputs a signal with a signal value in accordance with a concentration of a specific gas, to which a first gas and a second gas are supplied, the first gas and the second gas being different in at least either of obtaining position and obtaining time, gas information based on a signal output by the sensor unit in a period in which the first gas is supplied to the sensor unit and a signal output by the sensor unit in a period in which the second gas is supplied to the sensor unit; and
a controller configured to generate health information using the gas information.

2. The health condition estimation apparatus according to claim 1, wherein:
a signal output by the sensor unit in the period in which the first gas is supplied to the sensor unit is a signal output by the sensor unit in each of time sections, which are obtained by dividing the period in which the first gas is supplied to the sensor unit into a plurality of time sections,
a signal output by the sensor unit in the period in which the second gas is supplied to the sensor unit is a signal output by the sensor unit in each of time sections, which are obtained by dividing the period in which the second gas is supplied to the sensor unit into a plurality of time sections, and
the gas information includes a signal value of a signal output by the sensor unit in each of the plurality of time sections, or at least either of at least one of a mean, median, and slope based on the signal value in each of the time sections, and a difference in at least one of the mean, median, and slope from a different time section.

3. The health condition estimation apparatus according to claim 2, wherein:
the sensor unit includes a plurality of sensors having different sensitivities to the concentration of the specific gas, and
the gas information includes a ratio of signal values of signals output by the plurality of sensors in each of the plurality of time sections.

4. The health condition estimation apparatus according to claim 1, wherein:
the specific gas includes at least one of hydrogen, carbon dioxide, methane, hydrogen sulfide, methyl mercaptan, dimethyl sulfide, carboxylic acid, and amine.

5. The health condition estimation apparatus according to claim 1, wherein:
the controller configured to generate the health information additionally based on a subject's physical information.

6. The health condition estimation apparatus according to claim 5, wherein:
the physical information includes at least one of sex, age, height, weight, and body fat percentage.

7. The health condition estimation apparatus according to claim 1, wherein:
the controller configured to generate the health information additionally based on a subject's food intake information.

8. The health condition estimation apparatus according to claim 7, wherein:
the food intake information includes at least one of a food ingested by the subject, and a nutrient contained in the ingested food.

9. The health condition estimation apparatus according to claim 8, wherein:
the nutrient includes at least one of dietary fiber, starch, an oligosaccharide, a carbohydrate digestive enzyme inhibitor, and a protein.

10. The health condition estimation apparatus according to claim 1, wherein:
the health information is at least one of a ratio of intestinal bacteria, a ratio of categories that classify intestinal bacteria, and a health advice based on an intestinal bacterial status.

11. The health condition estimation apparatus according to claim 1, wherein:
in a case where a subject's intestinal bacterial status is obtained, the controller associates the intestinal bacterial status with identification information of the subject.

12. The health condition estimation apparatus according to claim 11, wherein:
the controller configured to give the intestinal bacterial status along with the identification information of the subject, which is associated with the intestinal bacterial status, to an analysis apparatus.

13. The health condition estimation apparatus according to claim 12, wherein:
the obtaining unit configured to obtain an estimation formula for calculating the health information at least based on the gas information, which is updated by the analysis apparatus.

14. The health condition estimation apparatus according to claim 11, wherein:
the controller configured to update an estimation formula for calculating the health information at least based on the gas information by conducting a multiple regression analysis based on the intestinal bacterial status associated with information used for generating the health information.

15. A health condition estimation method comprising:
separately supplying a first gas and a second gas to a sensor unit that outputs a signal with a signal value in accordance with a concentration of a specific gas, the first gas and the second gas being different in at least either of obtaining position and obtaining time; and
generating health information using gas information based on a signal output by the sensor unit in a period in which the first gas is supplied to the sensor unit and a signal output by the sensor unit in a period in which the second gas is supplied to the sensor unit.
